(19) United States
(12) Patent Application Publication
(10) Pub. No.: US 2011/0313343 A1
(43) Pub. Date: Dec. 22, 2011

(54) Phacoemulsification Fluidics System Having a Single Pump Head

(75) Inventors: Ivan Milutinovic, Irvine, CA (US); Gary P. Sorensen, Laguna Niguel, CA (US); Raphael Gordon, Ladera Ranch, CA (US); Dan Teodorescu, Fountain Valley, CA (US)

(73) Assignee: ALCON RESEARCH, LTD., Fort Worth, TX (US)

(21) Appl. No.: 12/818,682

(22) Filed: Jun. 18, 2010

(51) Int. Cl. A61F 9/007; A61M 1/00

(52) U.S. Cl. 604/22; 604/28; 604/30

(57) ABSTRACT

A phacoemulsification fluidics system for irrigating and aspirating a surgical site includes a sterile solution reservoir, an irrigation path configured to extend from the sterile solution reservoir to the surgical site, and an aspiration path configured to extend from the surgical site. The system also includes a single flow control pump head associated with both the irrigation path and the aspiration path. The flow control pump head is arranged within the system to simultaneously pressurize the irrigation path in a manner that drives the irrigation fluid to the surgical site and pressurize the aspiration path in a manner that vacuums waste fluid from the surgical site.

### BACKGROUND OF THE INVENTION

The present invention relates to phacoemulsification systems and more particularly to a system for regulating pressures in the eye during phacoemulsification surgeries.

In the United States, the majority of surgically treated cataractous lenses are treated by a surgical technique called phacoemulsification. A typical surgical hand piece suitable for phacoemulsification procedures consists of an ultrasonically driven phacoemulsification hand piece, an attached hollow cutting needle surrounded by an irrigating sleeve, and an electronic control console. The hand piece assembly is attached to the control console by an electric cable and flexible tubing. Through the electric cable, the console varies the power level transmitted by the hand piece to the attached cutting needle. The flexible tubing supplies irrigation fluid to the surgical site and draws aspiration fluid from the eye through the hand piece assembly.

During a phacoemulsification procedure, the tip of the cutting needle and the end of the irrigation sleeve are inserted into the anterior segment of the eye through a small incision in the eye's outer tissue. The surgeon brings the tip of the cutting needle into contact with the lens of the eye, so that the vibrating tip fragments the lens. The resulting fragments are aspirated out of the eye through the interior bore of the cutting needle, along with irrigation fluid provided to the eye during the procedure, and into a waste reservoir.

Throughout the procedure, irrigating fluid is pumped into the eye, passing between the irrigation sleeve and the cutting needle and exiting into the eye at the tip of the irrigation sleeve and/or from one or more ports or openings formed into the irrigation sleeve near its end. This irrigating fluid is critical, as it prevents the collapse of the eye during the removal of the emulsified lens. The irrigating fluid also protects the eye tissues from the heat generated by the vibrating of the ultrasonic cutting needle. Furthermore, the irrigating fluid suspends the fragments of the emulsified lens for aspiration from the eye.

Conventional systems employ fluid-filled bottles or bags hung from an IV pole as an irrigation fluid source. Irrigation flow rates, and corresponding fluid pressure at the eye, are regulated by controlling the height of the IV pole above the surgical site. For example, raising the IV pole results in a corresponding increase in irrigation flow rate and a corresponding increase in fluid pressure at the eye. Likewise, lowering the IV pole results in a corresponding decrease in the irrigation flow rate and a corresponding lower pressure at the eye.

Aspiration flow rates of fluid from the eye are typically regulated by an aspiration pump in fluid communication with the aspirating interior bore of the cutting needle. The aspiration flow is monitored to control the pump and regulated to achieve a proper balance with the irrigation flow in an effort to maintain a relatively consistent fluid pressure at the surgical site within the eye.

While a consistent fluid pressure in the eye is desirable during the phacoemulsification procedure, common occurrences and complications create fluctuations in fluid flow and pressure at the eye. For example, varying flow rates result in varying pressure losses in the irrigation fluid path from the irrigation fluid supply to the eye, thus causing changes in pressure in the anterior chamber (also referred to as Intra-Ocular Pressure or IOP). Higher flow rates result in greater pressure losses and lower IOP. As IOP lowers, the operating space within the eye diminishes.

Blockages or occlusions of the aspirating needle also are common occurrences and procedural techniques affecting the fluid pressure at the eye during the phacoemulsification process. As the irrigation fluid and emulsified tissue are aspirated away from the interior of the eye through the hollow cutting needle, pieces of tissue that are larger than the diameter of the needle's bore may occlude the needle's tip. While the tip is occluded, vacuum pressure builds up within the tip. The drop in pressure in the anterior chamber in the eye, caused by a relatively large quantity of fluid and tissue to be aspirated out of the eye too quickly when the occlusion is removed, can potentially result in eye collapse and/or cause the lens capsule to be torn.

Various techniques have been designed to control the pressures at the eye and to reduce the surge during a phacoemulsification process. However, there remains a need for improved phacoemulsification devices that maintain a stable IOP throughout varying flow conditions. The present disclosure is directed to addressing one or more of the deficiencies in the prior art.

### SUMMARY OF THE INVENTION

In one embodiment consistent with the principles of the present invention, the present invention is a phacoemulsification fluidics system for irrigating and aspirating a surgical site. The system includes a sterile solution reservoir, an irrigation path configured to extend from the sterile solution reservoir to the surgical site, and an aspiration path configured to extend from the surgical site. The system also includes a single flow control pump head associated with both the irrigation path and the aspiration path. The flow control pump head is arranged within the system to simultaneously pressurize the irrigation path in a manner that drives the irrigation fluid to the surgical site and pressurize the aspiration path in a manner that vacuums waste fluid from the surgical site.

In another embodiment consistent with the principles of the present invention, the present invention is a phacoemulsification fluidics system for irrigating and aspirating a surgical site. The system includes an irrigation path configured to extend to the surgical site, an aspiration path configured to extend from the surgical site, and a control system configured to regulate fluid flow to the surgical site. The control system includes a flow control pump head associated with both the irrigation path and the aspiration path. The flow control pump head is configured to simultaneously pump fluid through both the irrigation path and the aspiration path. The control system also includes at least one flow control shunt valve configured to control flow through at least one of the irrigation and aspiration path and at least one sensor configured to detect a parameter of fluid in at least one of the irrigation and aspiration paths. The control system also includes a controller in communication with the flow control pump head, the at least one flow control shunt valve, and the at least one sensor. The controller is structurally configured to receive data indicative of the detected parameter from the at least one sensor and structurally arranged to communicate control signals to the at least one flow control shunt valve based on the received data from the at least one sensor.

In another embodiment consistent with the principles of the present invention, the present invention is a phacoemulsification surgical console. The console includes an ultrasonic generator subsystem comprising an ultrasonic oscillation handpiece including a cutting needle. The handpiece is configured to emulsify a lens in an eye. The console also includes a fluidics subsystem. The fluidics subsystem includes a sterile solution reservoir, an irrigation path associated with the ultrasonic oscillation handpiece and configured to extend from the sterile solution reservoir to the surgical site, and an aspiration path associated with the ultrasonic oscillation handpiece and configured to extend from the surgical site. The fluidics subsystem also includes a single peristaltic pump head associated with both the irrigation path and the aspiration path. The peristaltic pump head is arranged within the system to pressurize the irrigation path in a manner that drives the irrigation fluid to the surgical site and being arranged within the system to create a vacuum in the aspiration path in a manner that vacuums waste fluid from the surgical site.

In one aspect consistent with the principles of the present invention, the present invention is a method of operating a fluidics subsystem of a phacoemulsification system. The method includes the steps of detecting a parameter of a fluid in an irrigation path of a phacoemulsification system, detecting a parameter of a fluid in an aspiration path of a phacoemulsification system, and controlling fluid flow through the irrigation and aspiration paths with a single flow control pump head associated with both the irrigation path and the aspiration paths.

It is to be understood that both the foregoing general description and the following detailed description are exemplary and explanatory only and are intended to provide further explanation of the invention as claimed. The following description, as well as the practice of the invention, sets forth and suggests additional advantages and purposes of the invention.

### BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 is an illustration of a phacoemulsification console including a single pump fluidics system that drives both irrigation and aspiration according to the principles of this disclosure.

FIG. 2 is a block diagram of the phacoemulsification console of FIG. 1 showing various subsystems including a single pump head fluidics system that drives both irrigation and aspiration according to the principles of the present disclosure.

FIG. 3 is a schematic of the fluidics subsystem from FIGS. 1 and 2 having the single pump head that drives both irrigation and aspiration according to the principles of the present disclosure.

FIG. 4 is a flow diagram of a control process for operating the single pump head fluidics system that drives both irrigation and aspiration according to the principles of the present disclosure.

FIG. 5 is a schematic of an alternative fluidics subsystem usable in the console of FIGS. 1 and 2 having the single pump head that drives both irrigation and aspiration according to the principles of the present disclosure.

FIG. 6 is a schematic of another alternative fluidics subsystem usable in the console of FIGS. 1 and 2 having the single pump head that drives both irrigation and aspiration according to the principles of the present disclosure.

FIG. 7 is a schematic of yet another alternative fluidics subsystem usable in the console of FIGS. 1 and 2 having the single pump head that drives both irrigation and aspiration according to the principles of the present disclosure.

FIG. 8 is a schematic of another alternative fluidics subsystem usable in the console of FIGS. 1 and 2 having the single pump head that drives both irrigation and aspiration according to the principles of the present disclosure.

### DETAILED DESCRIPTION OF THE PREFERRED EMBODIMENTS

Reference is now made in detail to the exemplary embodiments of the invention, examples of which are illustrated in the accompanying drawings.

Wherever possible, the same reference numbers are used throughout the drawings to refer to the same or like parts.

The phacoemulsification systems and methods described herein provide and control both irrigation and aspiration during an emulsification procedure with a single flow control pump head. These systems and methods provide independent control of positive irrigation pressure and negative aspiration pressure while simplifying product manufacturing and reducing manufacturing costs, while providing simplicity and effective control without compromising surgical results.

In addition, during periods of pressure variations, including for example, during needle occlusion or leakage, the systems and methods described herein compensate for these variations. Particularly, as described below with reference to the examples herein, a controller and control shunt valves recirculate or drain excess fluid in a manner that compensates for these pressure variations in the irrigation and aspiration flow paths. Accordingly, the systems and methods disclosed herein provide a level of consistency and repeatability while maintaining control of the system to achieve satisfactory surgical results.

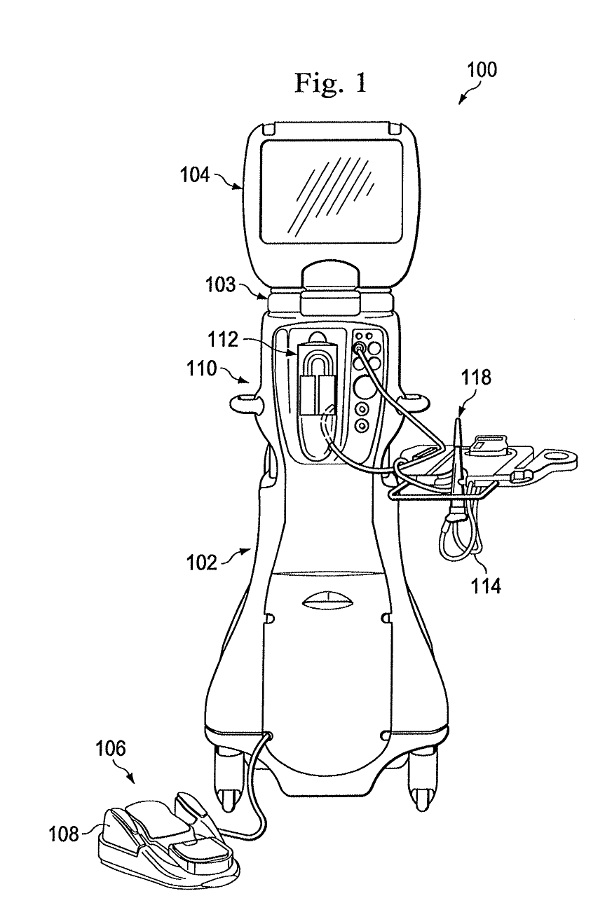

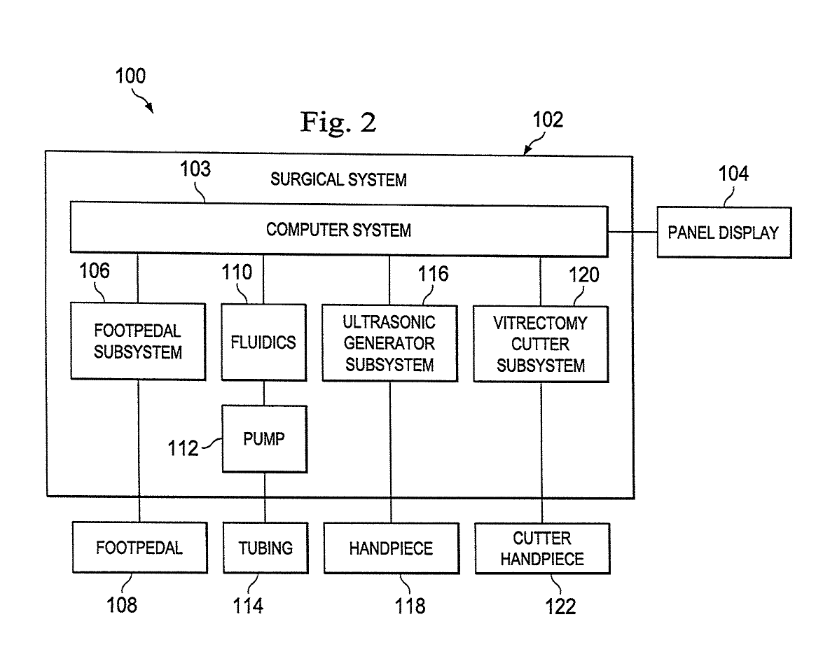

FIG. 1 illustrates an exemplary emulsification surgical console, generally designated 100. FIG. 2 is a block diagram of the console 100 showing various subsystems that operate to perform a phacoemulsification procedure. The console 100 includes a base housing 102 with a computer unit 103 and an associated display screen 104 showing data relating to system operation and performance during an emulsification surgical procedure. The console also includes a number of subsystems that are used together to perform the emulsification surgical procedures. For example, the subsystems include a footpedal subsystem 106 including, for example, a footpedal 108, a fluidics subsystem 110 including a single flow control pump 112 that both irrigates and aspirates the eye through flexible tubing 114, an ultrasonic generator subsystem 116 including an ultrasonic oscillation handpiece 118 with a cutting needle, and a pneumatic vitrectomy cutter subsystem 120 including a vitrectomy handpiece 122. These subsystems overlap and cooperate to perform various aspects of the procedure. For example, in some embodiments, the end of the flexible irrigation tubing is disposed about the cutting needle to provide irrigation and cooling to the cutter and tissue during the procedure. In addition, in some embodiments, an end of the flexible aspiration tubing is associated with the cutter needle and aspirated through a hollow bore in the cutter needle.

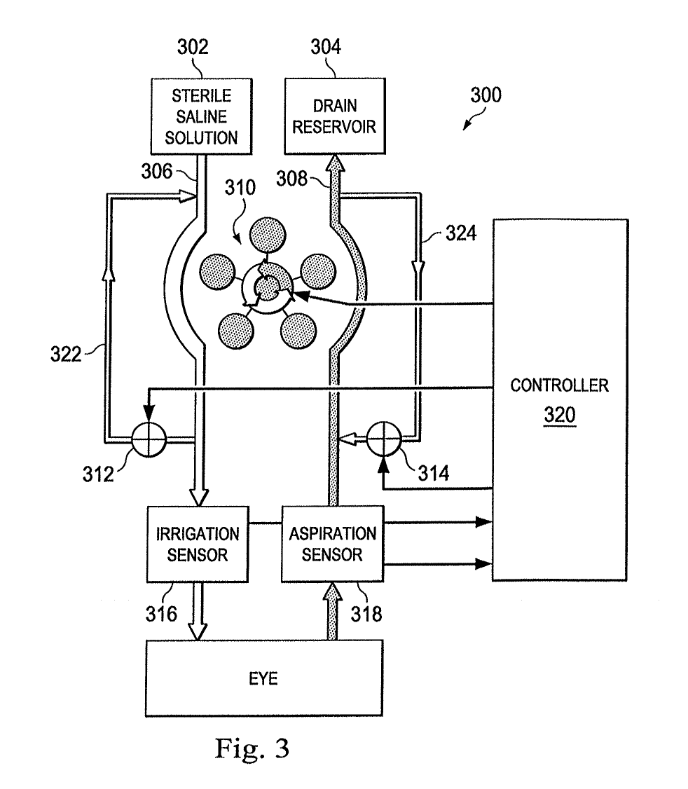

FIG. 3 illustrates a portion of the fluidics subsystem 110. It includes a flow control system 300, a sterile solution reservoir 302, a drain reservoir 304, an irrigation path 306, an aspiration path 308. The flow control system 300 includes a single flow control pump head 310, an irrigation flow control shunt valve 312, an aspiration flow control shunt valve 314, an irrigation pressure sensor 316, an aspiration pressure sensor 318, and a controller 320.

The irrigation path 306 extends between the sterile solution reservoir 302 and the surgical site (labeled in FIG. 3 as an eye) and carries sterile fluid from the reservoir 302 to the eye. In one example, the sterile fluid is a saline fluid, however, other fluids may be used. At least a portion of the irrigation path 306 may be formed of a flexible tubing. In some embodiments, the path 306 is formed of multiple segments, with some segments being rigid and others being flexible. In some embodiments, at least a portion of the irrigation path is formed in a cassette that cooperates with the console 100 in FIG. 1 to provide fluid communication between the sterile solution reservoir 302 and the patient's eye. As indicated above, in some embodiments, the end of the irrigation path 306 is disposed about the cutting needle to provide irrigating fluid flow to the eye during the surgical procedure. The irrigation path 306 in FIG. 3 is represented by a series of arrows showing the flow direction from the reservoir 302 to the eye.

The aspiration path 308 extends from the surgical site or eye to the drain reservoir 304. The aspiration path 308 carries away fluid used to flush the eye as well as any emulsified particles. Like the irrigation path, in FIG. 3, the aspiration path 308 is represented by a series of arrows showing the flow direction from the eye to the drain reservoir 304. Here, it is represented by the shaded arrows. As described above with reference to the irrigation path, at least a portion of the aspiration path 308 may be formed of a flexible tubing. In some embodiments, the path 308 is formed of multiple segments, with some segments being rigid and others being flexible. In some embodiments, at least a portion of the aspiration path 308 is formed in a cassette that cooperates with the console 100 in FIG. 1 to provide fluid communication between the patient's eye and the drain reservoir 304. It should be apparent that the drain reservoir 304 may in fact be a drain instead of a self-contained reservoir. As indicated above, in some embodiments, the aspiration path is in fluid communication with the bore of the cutter needle and is used to aspirate fluid and emulsified particles through the needle bore and into the aspiration path 308 during the surgical procedure.

In some embodiments, the fluidics system 110 is arranged to provide a higher fluid volume along the irrigation path 306 than along the aspiration path 308. This may be accomplished in a variety of ways, including for example, using a larger diameter fluid line in the irrigation path than a fluid line in the aspiration path as is shown in FIG. 3.

The single flow control pump head 310 is associated with both the irrigation and aspiration paths 306, 308. In the embodiment shown, the pump head operates in a manner that pumps fluid at an equal motor rate through both the irrigation path 306 and the aspiration path 308. In the embodiment disclosed herein, the flow control pump head 310 is a peristaltic pump head, and more particularly, a rotary peristaltic pump head having rollers that induce fluid flows in both the irrigation and aspiration paths 306, 308 to simultaneously pump fluid at the same speed through both paths. In the embodiment shown, the pump head 310 is configured to provide feedback data indicative of the speed of its operation. This feedback may be used to further control the pump to provide a desired fluid flow through the irrigation and aspiration paths 306, 308.

The irrigation and aspiration sensors 316, 318 perform the function of detecting any high pressure or vacuum conditions in the irrigation and aspiration paths 306, 308, respectively. In some embodiments, the sensors 316, 318 are pressure sensors configured to detect current pressure conditions. These sensors 316, 318 may communicate signals indicative of the sensed pressures to the controller 320. Once received, the controller 320 processes the received signals to determine whether the pressure is above or below pre-established desired thresholds, or within a pre-established desired range. Although described as pressure sensors, the irrigation and aspiration pressure sensors 316, 318 may be other types of sensors, such as flow sensors that detect actual flow past the sensors and may include additional sensors for monitoring additional parameters. In some embodiments each sensor includes its own processing function and the processed data is then communicated to the controller 320.

With reference to FIG. 3, the irrigation path is in communication with a pressure relief line 322. The pressure relief line 322 fluidly communicates with either the sterile solution reservoir 302 or a segment of the irrigation path 306 above the pump head 310. In use, the irrigation flow control shunt valve 312 may be actuated to vary fluid flow from the irrigation path 306 through the pressure relief line 322 when undesired pressure levels are detected at the irrigation pressure sensor 316.

Similarly, the aspiration path 308 is in communication with a vacuum pressure relief line 324. In the embodiment shown, the vacuum pressure release line 324 fluidly communicates with the aspiration path 308 to draw additional fluid between the eye and the pump head 310 to vary the fluid flow from the eye. In use, the aspiration flow control shunt valve 314 may be actuated to vary fluid flow into the aspiration path 308 from the vacuum relief line 324 when undesired vacuum pressure levels are detected at the aspiration pressure sensor 318.

The irrigation and aspiration flow control shunt valves 312, 314 are respectively associated with the pressure relief line 322 and the vacuum pressure relief line 324 and regulate the pressure in the irrigation and aspiration paths 306, 308. Accordingly, the irrigation and aspiration flow control shunt valves 312, 314 are associated with the irrigation and aspiration paths 306, 308 in a manner that controls fluid flow and modifies the fluid pressure in those paths. In some embodiments, the shunt valves 312, 314 are adjustable valves, although other valve types may be used. The first and second flow control shunt valves 312, 314 communicate with and are controlled by the controller 320 in order to provide desired fluid flow to the surgical site.

The controller 320 may include a processor and memory and may be configured or programmed to control the flow control system 300 based upon pre-established programs or sequences. In addition to controlling the flow control system 300, the controller 320 may cooperate with the footpedal subsystem 106 or other subsystem in FIG. 2 and may control some aspects of the flow control system 300 based upon data or signals received from these other subsystems.

In use, the controller 320 is configured to receive signals from the irrigation and aspiration pressure sensors 316, 318, and process the signals to determine whether the detected parameters are outside of preset acceptable ranges or above or below preset acceptable thresholds. Based upon the received signals, the controller 320 controls the irrigation and aspiration flow control shunt valves 312, 314 to increase or decrease flow through the relief lines 322, 324 to either maintain or adjust the pressures in the irrigation and aspiration paths 306, 308 to the desired levels. In some embodiments, the controller 320 also controls the flow control pump head 310 based on preset instructions. In some embodiments, the pump head is controlled based upon the data gathered by the irrigation and aspiration pressure sensors 316, 318 and/or any of the other subsystems in FIG. 2.

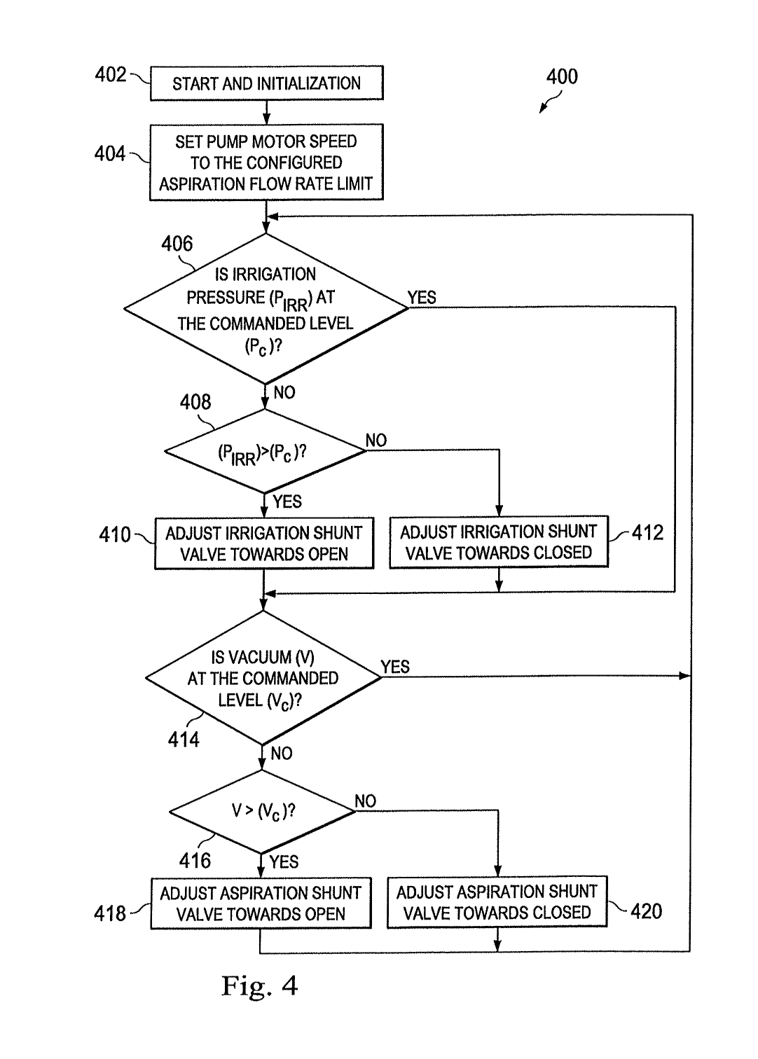

FIG. 4 is an exemplary control process 400 executable by the controller 320 for controlling the fluidics subsystem 110 during a phacoemulsification procedure. The process 400 begins at a start and initialization step 402. At step 404, the controller 320 sets the pump motor speed to the configured aspiration flow rate limit. The configured aspiration flow rate limit is a value set by the user via on-screen interface controls or the foot pedal 108, or combination of both. This set value is determined by the user to be satisfactory for the surgical procedure. Once the pump speed is driving fluid flow through the system, the interaction between the measured pressures, the commanded pressures, and the shunt valves will be monitored and controlled.

At a step 406, the controller 320 determines whether the irrigation pressure in the irrigation line 306 is at the commanded level. The irrigation pressure is detected by the irrigation pressure sensor 316. The commanded level is the level corresponding to a desired pressure set by the user. If the irrigation pressure is not at the commanded level at step 406, then the controller 320 is configured to control the fluidics subsystem 110 correct the deviation between the irrigation pressure and the commanded level. To do this, at a step 408, the controller 320 compares the detected irrigation pressure to the commanded pressure to determine whether the irrigation pressure is greater than the commanded pressure. In the embodiment described, this is accomplished by comparing signals or data obtained by and communicated from the irrigation pressure sensor 316 to the controller 320 with the user setting stored in the controller 320.

If the irrigation pressure is greater than the commanded pressure, then at step 410, the controller adjusts the irrigation flow control shunt valve 312 to increase the state or adjust toward a more open position, thereby permitting some fluid flow in the irrigation line to shunt into the pressure relief line 322. This decreases the percentage of total flow directed to the irrigation pressure sensor 316 and the surgical site and simultaneously increases the percentage of fluid flow flowing through the pressure relief line 322. Decreasing the total irrigation flow towards surgical site results in decreased fluid pressure at the surgical site.

If the irrigation pressure is less than the commanded pressure at step 408, then the controller 320 controls the irrigation flow control shunt valve 312 to decrease the state or adjust to a more closed position at step 416. This increases the percentage of total fluid flow being directed to the irrigation pressure sensor 316 and the surgical site. It simultaneously decreases the percentage of fluid flow flowing through the pressure relief line 322. Increasing the total irrigation flow towards the surgical site results in a higher fluid pressure at the surgical site. After adjusting the irrigation shunt valve at either step 410 or step 412, the method proceeds to step 414.

Returning to step 406, if the irrigation pressure is at the commanded level, then the method proceeds to step 414.

At step 414, the controller 320 determines whether the vacuum pressure in the aspiration path 308 is at the vacuum commanded level. The vacuum pressure is detected by the aspiration pressure sensor 318. The vacuum commanded level is the level corresponding to a desired input set by the user via the on-screen interface controls or the foot pedal 108, or combination of both. If the vacuum pressure is not at the commanded level at step 414, then the controller 320 is configured to control the fluidics subsystem 110 correct the deviation between the vacuum pressure and the commanded level. To do this, at a step 416, the controller 320 compares the detected vacuum pressure to the commanded vacuum pressure to determine whether the vacuum pressure is greater than the commanded vacuum pressure. In the embodiment described, this is accomplished by comparing signals or data obtained by and communicated from the aspiration pressure sensor 318 to the controller 320 with the user setting stored in the controller 320.

If the vacuum is greater than the commanded vacuum level at step 416, then the controller 320 controls the aspiration flow control shunt valve 314 to increase the state or adjust to a more open position at step 418. This decreases the percentage of total flow from the surgical site and simultaneously increases the percentage of fluid flow being drawn from the pressure relief line 324. Drawing less fluid directly from the surgical site results in an increase in the overall pressure (and a decreased vacuum) being detected by the aspiration pressure sensor 318.

If the vacuum is not greater than the commanded vacuum level at step 416, then the controller 320 controls the aspiration flow control shunt valve 314 to decrease the state or adjust the aspiration flow control shunt valve 314 to a more closed position at step 420. This increases the percentage of total fluid flow being drawn from the surgical site, and simultaneously decreases the percentage of fluid flow being drawn from the pressure relief line 324. Drawing more fluid directly from the surgical site results in a decrease in the overall pressure (and an increased vacuum) being detected by the aspiration pressure sensor 318.

Returning to step 414, if the vacuum pressure is at the commanded level, then the method returns to step 406 to monitor and control the irrigation shunt valve. Thus, the described process acts as an infinite loop by returning to step 406, such that the controller 320 continuously control the irrigation and aspiration flow control shunt valves 312, 314 based on the data from the irrigation and aspiration pressure sensors 316, 318.

One skilled in the art will recognize that additional flexibility may be achieved by controlling the pump motor speed along with controlling the shunt valves to increase or decrease flow and pressures in the irrigation and aspiration lines.

As described above, in some embodiments, the system is arranged to have more fluid than surgically necessary drawn through the irrigation path 306. It also may be arranged to draw more fluid through the irrigation path 306 than through the aspiration path 308. By drawing excess fluid through the irrigation path 306, the irrigation flow control shunt valve 312 may be continuously maintained in a partially open condition, thereby continuously being able to be controlled to increase or decrease fluid flow through the pressure relief line to vary the pressure in the irrigation path 306. Further, the system can therefore compensate for variations in the pressures caused by changes in flow rate, occlusions, or leakage of the fluid from the surgical site or else respond to changes in set pressure based on user inputs. These variations typically cause corresponding variations in the pressure levels of the irrigation and aspiration paths. Controlling the flow control shunt valves 312, 314 based on the detected pressures decreases the chance of complications resulting in the collapse of the eye.

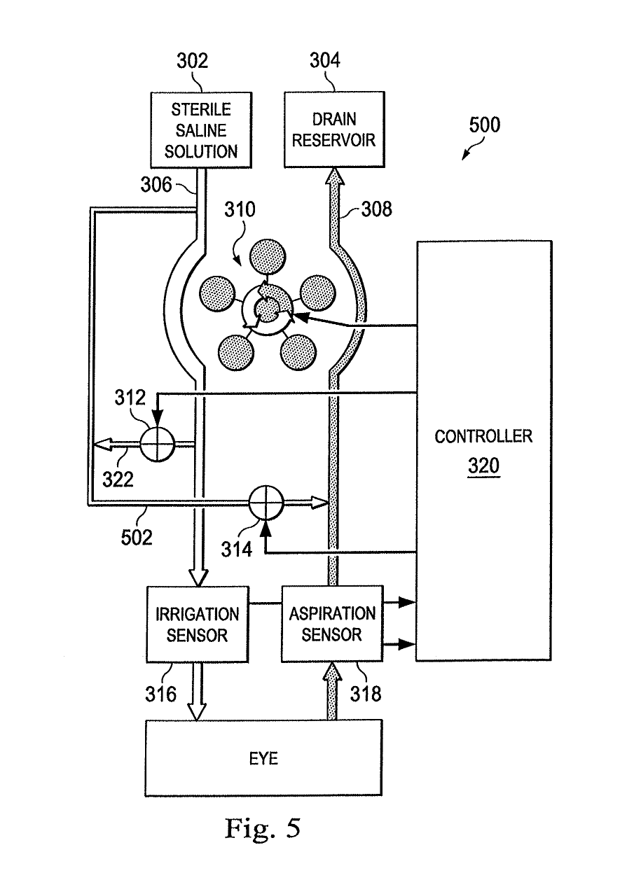

FIG. 5 shows an alternative arrangement of a portion of a fluidics subsystem 500 using a single flow control pump head 310 to drive both the irrigation and aspiration paths. Many elements of the fluidics system in FIG. 5 are the same as or similar to the elements of the fluidics system in FIG. 3. In order to avoid redundancy, explanations of these common elements are not repeated here. FIG. 5 includes the irrigation path 306, the aspiration path 308, and the flow control shunt valves 312, 314. As can be seen however, FIG. 5 includes a vacuum pressure relief line 502 that connects to the sterile solution source, such as the irrigation path 306 above the pump head 310. In other embodiments, the vacuum pressure relief line connects to the sterile solution reservoir 302. Accordingly, controlling the flow control shunt valve 314 adjusts the amount of fluid being allowed from the sterile solution source to the aspiration path 308, thereby providing control of the pressure in the aspiration path 308.

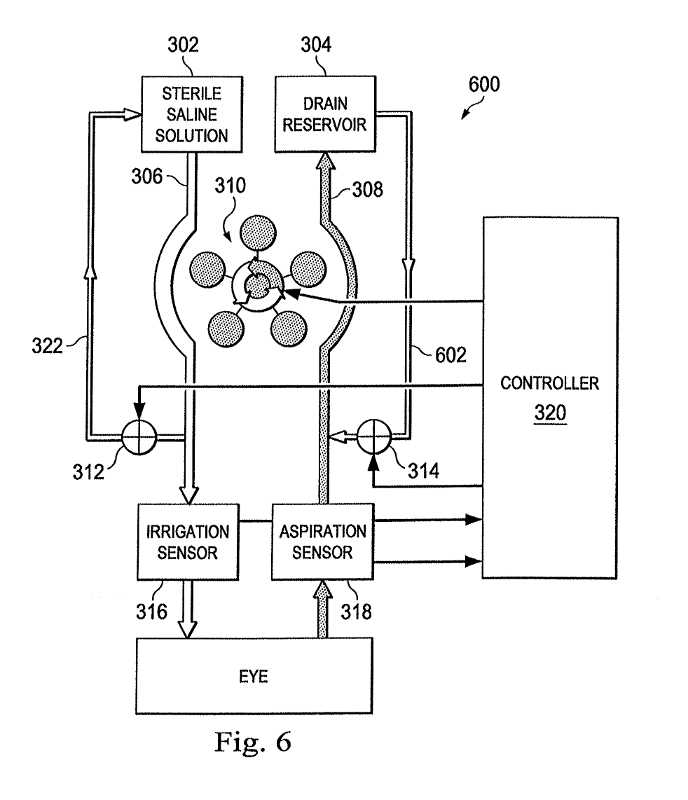

FIG. 6 shows another alternative arrangement of a portion of a fluidics subsystem 600 using a single flow control pump head to drive both the irrigation and aspiration paths. Many elements of the fluidics system in FIG. 6 are the same as or similar to the elements of the fluidics system in FIG. 3. In order to avoid redundancy, explanations of these common elements are not repeated here. FIG. 6 includes the irrigation path 306, the aspiration path 308, and the flow control shunt valves 312, 314. In FIG. 6, a vacuum pressure release line 602 fluidly communicates with the drain reservoir 304. This contrasts with FIG. 3 where the vacuum pressure relief line 324 in FIG. 3 communicates with the sterile aspiration path 324 above the pump head 308. In use, the aspiration flow control shunt valve 314 is actuated to permit fluid flow into the aspiration path 308 from the vacuum relief line 602 when undesired vacuum pressure levels are detected at the aspiration pressure sensor 318.

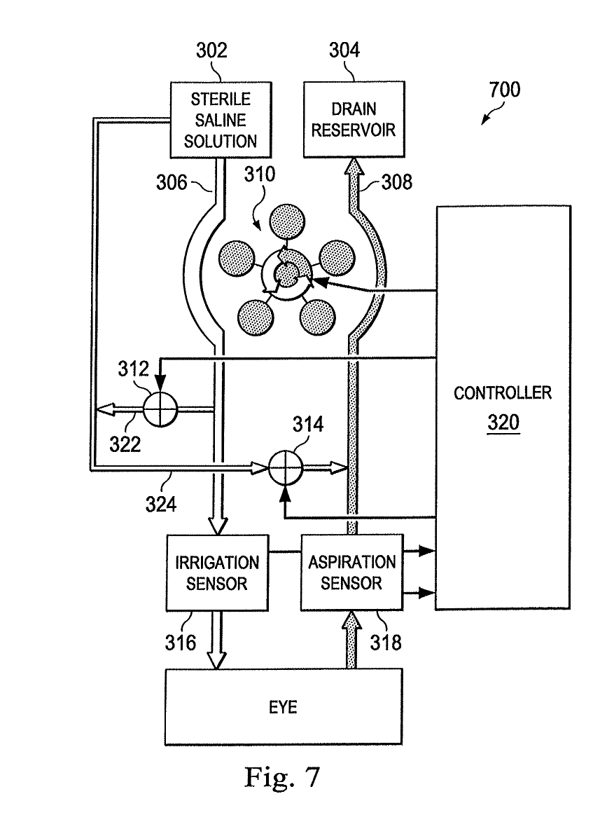

FIG. 7 shows an alternative arrangement of a portion of a fluidics subsystem 700 using a single flow control pump head 310 to drive both the irrigation and aspiration paths. The system 7 differs from system 500 in FIG. 5 only where the pressure relief lines 322, 324 connect to the sterile solution reservoir 302 instead of the irrigation path 306 above the pump head 310. Controlling the flow control shunt valves 312, 314 adjusts the amount of fluid being drawn directly from the sterile solution source 302, thereby providing control of the pressure in the irrigation and aspiration paths 306, 308.

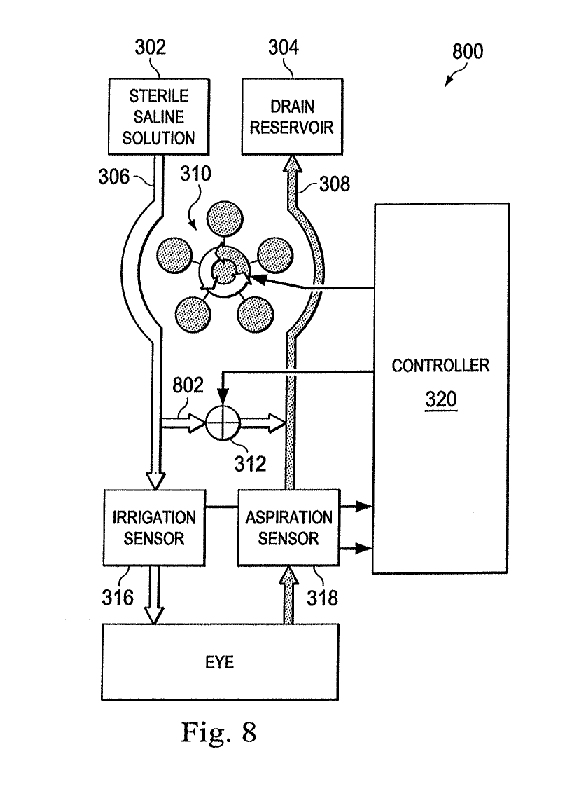

FIG. 8 shows an alternative arrangement of a portion of a fluidics subsystem 800 using a single flow control pump head 310 to drive both the irrigation and aspiration paths. Many elements of the fluidics system in FIG. 8 are the same as or similar to the elements of the fluidics system in FIG. 3. In order to avoid redundancy, explanations of these common elements are not repeated here. FIG. 8 includes the irrigation path 306 and the aspiration path 308. As can be seen however, FIG. 8 does not include pressure relief lines. Instead, FIG. 8 includes a single relief line 802 extending between the irrigation and aspiration paths 306, 308. Flow through the line 802 is controlled by the a flow control shunt valves 312. Accordingly, changing the state or adjusting the flow control shunt valve 312 may simultaneously affect the pressure in both the irrigation and aspiration paths.

It should be appreciated that although several different embodiments are shown, any of the features of one embodiment may be used on any of the other embodiments shown. Accordingly, any of these embodiments may include relief lines that extend to the solution reservoirs or to a fluid line or path. In some embodiments, the relief lines connect to the fluid paths near the pump head. In embodiments using a cassette, the relief lines may also be included within the cassette itself. In addition, while several embodiments are shown, still others are contemplated that include alternative arrangements of the shunt valves and connection locations of the relief lines.

From the above, it may be appreciated that the present invention provides a fluidics system having a single pump head irrigation and aspiration system for phacoemulsification surgery.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A phacoemulsification fluidics system for irrigating and aspirating a surgical site, comprising:
a sterile solution reservoir;
an irrigation path configured to extend from the sterile solution reservoir to the surgical site;
an aspiration path configured to extend from the surgical site; and
a single flow control pump head associated with both the irrigation path and the aspiration path, the flow control pump head being arranged within the system to simultaneously pressurize the irrigation path in a manner that drives the irrigation fluid to the surgical site and pressurize the aspiration path in a manner that vacuums waste fluid from the surgical site.

2. The phacoemulsification fluidics system of claim 1, further comprising:
an irrigation flow control shunt valve fluidly associated with the irrigation path and configured to change the pressure in the irrigation path; and
an aspiration flow control shunt valve fluidly associated with the aspiration path and configured to change the pressure in the aspiration path.

3. The phacoemulsification fluidics system of claim 2, further comprising a controller in communication with the irrigation and aspiration flow control shunt valves, the controller regulating the shunt valves to maintain a preset pressure within the respective irrigation path and aspiration path.

4. The phacoemulsification fluidics system of claim 2, further comprising a pressure relief line connecting the irrigation flow control shunt valve to the sterile solution reservoir, the flow control shunt valve being arranged to control fluid flow from the flow control pump head to the pressure relief line when pressure in the irrigation exceeds a pre-established level.

5. The phacoemulsification fluidics system of claim 4, further comprising a vacuum pressure relief line connecting the aspiration flow control shunt valve to one of a fluid source and a drain, the aspiration flow control shunt valve being arranged to control fluid from the vacuum pressure relief line to the aspiration path when pressure in the aspiration path falls below a pre-established level.

6. The phacoemulsification fluidics system of claim 1, further comprising:
an irrigation pressure sensor associated with the irrigation path and configured to detect a parameter of the fluid within the irrigation path; and
an aspiration pressure sensor associated with the aspiration path and configured to detect a parameter of the fluid within the aspiration path.

7. The phacoemulsification fluidics system of claim 6, wherein the irrigation and aspiration pressure sensors comprise pressure sensors arranged to detect pressure within the respective irrigation and aspiration paths.

8. The phacoemulsification fluidics system of claim 1, wherein the single flow control pump head is a head of a peristaltic pump directing fluid at the same motor speed through the irrigation path to the surgical site and from the surgical site through the aspiration path.

9. The phacoemulsification fluidics system of claim 1, comprising a pressure relief line connecting the irrigation path and the aspiration path.

10. The phacoemulsification fluidics system of claim 1, wherein the flow control pump head is configured to pump fluid at the same motor speed through both the irrigation path and the aspiration path.

11. A phacoemulsification fluidics system for irrigating and aspirating a surgical site, comprising:
an irrigation path configured to extend to the surgical site;
an aspiration path configured to extend from the surgical site; and
a control system configured to regulate fluid flow to the surgical site comprising:
a flow control pump head associated with both the irrigation path and the aspiration path, the flow control pump head being configured to simultaneously pump fluid through both the irrigation path and the aspiration path,
at least one flow control shunt valve configured to control flow through at least one of the irrigation and aspiration paths;
at least one sensor configured to detect a parameter of fluid in at least one of the irrigation and aspiration paths;
a controller in communication with the flow control pump head, the at least one flow control shunt valve, and the at least one sensor, the controller being structurally configured to receive data indicative of the detected parameter from the at least one sensor, the controller also being structurally arranged to communicate control signals to the at least one flow control shunt valve based on the received data from the at least one sensor.

12. The phacoemulsification fluidics system of claim 11, wherein the at least one flow control shunt valve comprises:
an irrigation flow control shunt valve configured to control flow through the irrigation path; and
an aspiration flow control shunt valve configured to control flow through the aspiration path.

13. The phacoemulsification fluidics system of claim 11, wherein the at least one sensor comprises:
an irrigation pressure sensor configured to detect a parameter of fluid in the irrigation path; and
an aspiration pressure sensor configured to detect a parameter of fluid in the aspiration path.

14. The phacoemulsification fluidics system of claim 13, wherein the irrigation and aspiration pressure sensors comprise pressure sensors arranged to detect pressure within the respective irrigation and aspiration paths.

15. The phacoemulsification fluidics system of claim 11, comprising:
a sterile solution reservoir; and
a pressure relief line connecting the irrigation flow control shunt valve to the sterile solution reservoir, the irrigation flow control shunt valve being arranged to control fluid flow from the flow control pump head to the pressure relief line when pressure in the irrigation exceeds a pre-established level.

16. The phacoemulsification fluidics system of claim 11, further comprising:
one of a fluid source and drain; and
a vacuum pressure relief line connecting the aspiration flow control shunt valve to the one of the fluid source and drain, the aspiration flow control shunt valve being arranged to control fluid flow from the vacuum pressure relief line when pressure in the aspiration path falls below a pre-established level.

17. A phacoemulsification surgical console comprising:
an ultrasonic generator subsystem comprising an ultrasonic oscillation handpiece including a cutting needle, the handpiece being configured to emulsify a lens in an eye; and
a fluidics subsystem comprising
a sterile solution reservoir;
an irrigation path associated with the ultrasonic oscillation handpiece and configured to extend from the sterile solution reservoir to the surgical site;
an aspiration path associated with the ultrasonic oscillation handpiece and configured to extend from the surgical site; and
a single peristaltic pump head associated with both the irrigation path and the aspiration path, the peristaltic pump head being arranged within the system to pressurize the irrigation path in a manner that drives the irrigation fluid to the surgical site and being arranged within the system to create a vacuum in the aspiration path in a manner that vacuums waste fluid from the surgical site.

18. The phacoemulsification surgical console of claim 18, comprising:
an irrigation flow control shunt valve associated with the irrigation path and disposed downstream of the peristaltic pump head;
an aspiration flow control shunt valve associated with the aspiration path and disposed upstream of the peristaltic pump head;
an irrigation pressure sensor associated with the irrigation path and configured to detect a parameter of the fluid within the irrigation path, the irrigation pressure sensor being disposed downstream of the irrigation flow control shunt valve;
an aspiration pressure sensor associated with the aspiration path and configured to detect a parameter of the fluid within the aspiration path, the aspiration pressure sensor being disposed upstream of the aspiration flow control shunt valve; and
a controller in communication with the irrigation and aspiration flow control shunt valves and in communication with the irrigation and aspiration pressure sensors, the controller being configured to receive information from the irrigation and aspiration pressure sensors and to send control signals to the irrigation and aspiration flow control shunt valves based on the received information to effect a pressure change within the respective irrigation path and aspiration path.

19. The phacoemulsification surgical console of claim 18, comprising:
one of a sterile solution reservoir and drain; and
a pressure relief line connecting the irrigation flow control shunt valve to the one of a sterile solution reservoir and drain, the irrigation flow control shunt valve being arranged to direct fluid from the peristaltic pump head to the pressure relief line when pressure in the irrigation exceeds a pre-established level.

20. The phacoemulsification surgical console of claim 18, further comprising:
a fluid source; and
a vacuum pressure relief line connecting the aspiration flow control shunt valve to the fluid source, the aspiration flow control shunt valve being arranged to direct fluid from the vacuum pressure relief line when pressure in the aspiration path falls below a pre-established level.

21. A method of operating a fluidics subsystem of a phacoemulsification system, comprising:
detecting a parameter of a fluid in an irrigation path of a phacoemulsification system;
detecting a parameter of a fluid in an aspiration path of a phacoemulsification system; and
controlling fluid flow through the irrigation and aspiration paths with a single flow control pump head associated with both the irrigation path and the aspiration path.

22. The method of claim 21, comprising:
adjusting the state of an irrigation flow control shunt valve associated with the irrigation path to control fluid flow into a pressure release line.

23. The method of claim 22, comprising:
adjusting the state of an aspiration flow control shunt valve associated with the aspiration path to control fluid flow into the aspiration path from a vacuum pressure release line.
